(19) United States
(12) Patent Application Publication
(10) Pub. No.: US 2011/0009654 A1
(43) Pub. Date: Jan. 13, 2011

(54) 11B-FLUORO-3-ACETOXYESTRA-3,5-DIEN-17-ONE AND METHOD FOR THE PRODUCTION THEREOF

(76) Inventors: Orlin Petrov, Berlin (DE); Matthias Schneider, Potsdam (DE); Rolf Bohlmann, Berlin (DE); Stephan Vettel, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/739,195

(22) PCT Filed: Oct. 18, 2008
(86) PCT No.: PCT/EP08/08833

§ 371 Date: Sep. 9, 2010

(30) Foreign Application Priority Data
Oct. 24, 2007 (EP) 07075924.6

(51) Int. Cl. C07J 75/00; C07J 1/00

(52) U.S. Cl. 552/623

(57) ABSTRACT

The present invention relates to 11β-fluoro-3-acetoxyestra-3,5-dien-17-one as a suitable intermediate in the preparation of 11-fluoro-substituted steroids and to the process for preparation thereof. For this purpose, 11α-hydroxyestra-4-ene-3,17-dione is reacted with 1 to 3 equivalents of n-nonafluorobutanesulfonyl fluoride and 3 to 5 equivalents of diazabicycloundecene (DBU) at −40 to −20° C. in an organic aprotic solvent and, after an aqueous workup, reacted with 5 to 10 equivalents of acetic anhydride and 0.01 to 1 equivalent of a strong acid. The desired product precipitates spontaneously out of the reaction solution and is obtained in a very high purity by filtration. The process is notable for the very high yield, avoidance of a chromatographic purification of the product, a reduced proportion of wastes and significantly increased process throughput. The process according to the invention is therefore especially suitable for preparing 11β-fluoro-3-acetoxyestra-3,5-dien-17-one on a large industrial scale.

The present invention relates to 11β-fluoro-3-acetoxyestra-3,5-dien-17-one (I). 11β-Fluoro-3-acetoxyestra-3,5-dien-17-one (I)

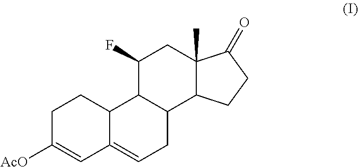

is suitable as an intermediate for the preparation of 11-fluoro-substituted steroids which find use as active pharmaceutical ingredients. The invention further relates to a process for preparation thereof.

11β-Fluoro-7α-substituted steroids are pharmacologically highly potent compounds with marked androgenic or antiestrogenic action. Examples includes the androgens described in WO 2004/011663 and WO 2002/059139, and the antiestrogens described in WO 03/045972, WO 99/33855 and WO 98/07740.

A key step in the synthesis of these compounds is the introduction of the 11β-fluoro substituent by a deoxyfluorination reaction of the 11α-hydroxy-substituted precursor (WO 2002/059139). An established method for direct conversion of primary and secondary alcohols including corresponding hydroxy steroids to the corresponding fluorides (deoxyfluorination) is described in the literature (e.g. H. Vorbrüggen et al., Tetrahedron Letters, 1995, 2611; J. Yin et al, Organic Letters 2004, 1465; Ch. Marson at al. Synthetic Communications 2002, 2125; U.S. Pat. No. 5,760,255, U.S. Pat. No. 6,248,889). In this method, the corresponding alcohol is reacted with commercially available n-nonafluorobutanesulfonyl fluoride and a strong organic base (preferably diazabicycloundecene (DBU)) in a suitable organic solvent (e.g. toluene, xylene, diglyme, dichloromethane, hexane, etc.) and, after aqueous workup, extraction and chromatography, the corresponding fluorine derivative is isolated.

U.S. Pat. No. 6,248,889 describes a deoxyfluorination process in which the use of a small or no excess of base is described as advantageous.

Vorbrüggen et al. (Tetrahedron Letters, 1995, 2611) describes specifically the conversion of 11α-hydroxy-19-norandrost-4-ene-3,17-dione (A) to the corresponding 11β-fluoro compound by reaction with 1.5 equivalents of n-nonafluorobutanesulfonyl fluoride and 3 equivalents of DBU in toluene at 24-30° C. After a chromatographic purification, 11β-fluoro-19-norandrost-4-ene-3,17-dione is obtained (B; example 1b), c) in WO 2002/059139) in a yield of 66% of theory.

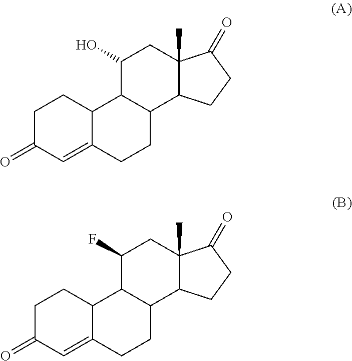

A similar reaction (74% yield after chromatography) with 1.5 equivalents of n-nonafluorobutanesulfonyl fluoride and 2.8 equivalents of DBU at 0° C. is described in DE 10104327.

Disadvantages of these methods are not only the moderate yields but the poor process throughputs, since large amounts of solvent are required both in the reaction and in the multiple extraction during the workup. In general, after the reaction workup, a crude product is obtained, the amount of which is 3 times greater than the amount of the starting material used. For isolation of the product from the crude mixture, a chromatographic purification is unavoidable. Such processes are therefore unsuitable for preparing 11β-fluorosteroids in multikilogram amounts.

It is therefore an object of the present invention to provide a process for preparing 11β-fluoro-3-acetoxyestra-3,5-dien-17-one (I), which permits simple recovery of the product and is notable for a good yield and good process throughput.

It has now been found in accordance with the invention that, after the temperature-controlled reaction of 11β-hydroxyestra-4-ene-3,17-dione (A) with 1.5-2 equivalents of n-nonafluoro-butanesulfonyl fluoride in the presence of an excess of at least 3.3 equivalents of DBU at −40 to −20° C., followed by a pH-controlled aqueous workup and subsequent acetylation of the intermediate, the 11β-fluoro-3-acetoxyestra-3,5-dien-17-one surprisingly crystallizes out of the reaction mixture spontaneously, and can be obtained in a very high yield (79-86% of theory) by simple filtration of the reaction mixture.

The solvents employed for the deoxyfluorination reaction may be aprotic solvents, for example methylene chloride, toluene, ethyl acetate, isopropyl acetate or benzotrifluoride. Preference is given to employing ethyl acetate.

For the second process step (acetylation reaction), acetic anhydride or isopropenyl acetate is used, or vinyl acetate in the presence of strong acids, for example p-toluenesulfonic acid (p-TsOH), methanesulfonic acid, sulfuric acid or hydrogen bromide (HBr).

Preference is given to using acetic anhydride in the presence of a catalytic amount of p-TsOH.

The 11β-fluoro-3-acetoxyestra-3,5-dien-17-one thus obtained in good yield and purity can be converted by hydrolysis in a bromation/dehydrobromination process known to those skilled in the art to the corresponding 11β-fluoro-19-norandrosta-4,6-diene-3,17-dione (WO 2002/059139; example 1c).

The latter can in turn be converted further by known processes to androgens or antiestrogens.

The examples which follow serve to illustrate the invention in detail. All temperatures are in degrees Celsius (uncorrected) and all amounts are reported in percent by weight, unless stated otherwise.

### EXAMPLES

### 11β-Fluoro-3-acetoxyestra-3,5-dien-17-one

1. A mixture of 50 g of 11α-hydroxyestra-4-ene-3,17-dione and 85.6 ml of DBU (3.3 eq.) in 250 ml of ethyl acetate is cooled to −35° C. to −40° C. A solution of 50 ml of n-nonafluoro-butanesulfonyl fluoride (1.6 eq.) in 100 ml of ethyl acetate is slowly added dropwise thereto at −35° C. with vigorous stirring. The reaction solution is stirred at −35° C. for 15 hours until, according to HPLC, the 11α-hydroxyestra-4-ene-3,17-dione content is <1%. This is followed by warming to −10° C., stirring for a further 60 min and admixing of the reaction with 20 ml of water.

The cooling is removed and the reaction mixture is admixed with 100 ml of 2N sulfuric acid. The mixture is stirred at 10° C. for a further 90 min. The phases are separated and the organic phase is admixed with 22 ml of 2N sulfuric acid in order to establish a pH of 2. The phases are separated again and the organic phase is washed with 50 ml of sat. NaHCO₃ solution and with 50 mol of sat. sodium chloride solution, and concentrated under reduced pressure to approx. 160 ml. The mixture is admixed twice with 200 ml each time of ethyl acetate and concentrated at 60° C. under reduced pressure to approx. 160 ml. This gives a stirrable crystal slurry in each case.

147.1 ml of acetic anhydride (9 eq.) are added at 20° C. to the stirrable crystal slurry. The mixture is cooled to 0° C. Within 4 h, a total of 2.24 ml of methanesulfonic acid (0.4 eq.) is added in 3 portions. The mixture is stirred at 0° C. for a further 44 hours and then the precipitated reaction product is filtered off with suction and washed four times with 40 ml of ice-cold isopropyl acetate and dried at 40° C. under reduced pressure.

This gives 49.6 g (86.1% of theory) of 11β-fluoro-3-acetoxyestra-3,5-dien-17-one as a pale beige, crystalline solid.

m.p.: 175-177° C.; [α]_{D}=−32.3° in CHCl₃; H NMR (δ in CHCl₃): 5.82 [1 H, d, J=2 Hz], 5.50 [1 H, m], 5.08 [1 H, d(br), J=49 Hz], 2.15 [3 H, s], 1.04 [3 H, d, J=1.5 Hz] [ppm].

2. 0.5 kg of 11α-hydroxyestra-4-ene-3,17-dione and 856 ml of DBU (3.3 eq.) are suspended in 3.25 l of ethyl acetate and cooled to −35 to −40° C. A solution of 500 ml of n-nonafluorobutanesulfonyl fluoride (1.6 eq.) in 250 ml of ethyl acetate is added thereto within 120 min.

After the addition has ended, the reaction solution is stirred at −35° C. for 6 hours. Thereafter, it is warmed to −10° C. within 30 min. The mixture is left to stir at −10° C. for another 120 min and the reaction is admixed with 600 ml of 2N sulfuric acid. The mixture is stirred for a further 60 min, in the course of which it is warmed to 30° C.

The phases are separated and the organic phase is admixed with 600 ml of 2N sulfuric acid in order to establish a pH of 3. The phases are separated again and the organic phase is washed with 800 ml of sat. sodium hydrogencarbonate solution and concentrated at 60° C. and 120 mbar to approx. 1.8 l. The mixture is admixed twice with 1.5 l each time of ethyl acetate and concentrated at 60° C. and 120 mbar to approx. 1.8 l. This forms a stirrable crystal slurry in each case, which is subsequently admixed with 1.471 l of acetic anhydride (9 eq.). The mixture is cooled to 0° C. and, after an addition of 132 g of p-toluenesulfonic acid (0.4 eq.), stirred at 0° C. for 24 hours. The precipitated reaction product is filtered off with suction, washed five times with 500 ml of ice-cold ethyl acetate and dried at 40° C. under reduced pressure.

This gives 475 g (82.5% of theory) of 11β-fluoro-3-acetoxyestra-3,5-dien-17-one as a pale beige, crystalline solid.

### Hydrolysis of 11β-fluoro-3-acetoxyestra-3,5-dien-17-one to 11β-fluoro-19-norandrost-4-ene-3,17-dione

950 g of 11β-fluoro-3-acetoxyestra-3,5-dien-17-one are dissolved in 10 l of methanol. 2.0 l of a saturated potassium carbonate solution are added thereto at 50° C. After 5 hours, the reaction mixture is cooled to room temperature and concentrated to approx. 3 l under reduced pressure. The precipitated product is filtered off, washed with water and dried. This gives 755 g (91% of theory) of 11β-fluoro-19-norandrost-4-ene-3,17-dione as a white solid. m.p. 171-173° C.

1. 11β-Fluoro-3-acetoxyestra-3,5-dien-17-one.

2. A process for preparing 11β-fluoro-3-acetoxyestra-3,5-dien-17-one, characterized in that 11α-hydroxyestra-4-ene-3,17-dione is allowed to react with 1 to 3 equivalents of n-nonafluorobutanesulfonyl fluoride and 3 to 5 equivalents of diazabicycloundecene (DBU) at −40 to −20° C. in an organic aprotic solvent and, after an aqueous intermediate workup, reacted with 5 to 10 equivalents of acetylating agent in the presence of 0.01 to 1 equivalent of a strong acid, and the precipitated product is obtained by filtering the reaction mixture.

3. The process for preparing 11β-fluoro-3-acetoxyestra-3,5-dien-17-one as claimed in claim 2, characterized in that more than 3 equivalents of DBU are used.

4. The process for preparing 11β-fluoro-3-acetoxyestra-3,5-dien-17-one as claimed in claim 2, characterized in that ethyl acetate is used as the solvent.

5. The process for preparing 11β-fluoro-3-acetoxyestra-3,5-dien-17-one as claimed in claim 2, characterized in that reaction is effected with acetic anhydride as the acetylating agent.

6. The process for preparing 11β-fluoro-3-acetoxyestra-3,5-dien-17-one as claimed in claim 2, characterized in that reaction is effected with p-tolulenesulfonic acid (p-TsOH) as the strong acid.
